(19) United States
(12) Patent Application Publication
(10) Pub. No.: US 2018/0100132 A1
(43) Pub. Date: Apr. 12, 2018

(54) Large Container

(71) Applicant: Planet Biogas Group GMBH, Vreden (DE)

(72) Inventor: Jörg Meyer Zu Strohe, Velen (DE)

(21) Appl. No.: 15/809,412

(22) Filed: Nov. 10, 2017

Related U.S. Application Data
(63) Continuation of application No. PCT/EP2016/057458, filed on Apr. 5, 2016.

(30) Foreign Application Priority Data
May 19, 2015 (DE) 20 2015 102 563.7

(51) Int. Cl. C12M 1/107; C12M 1/34; C12M 1/00; B65D 90/02

(52) U.S. Cl. CPC C12M 21/04; C12M 41/12; B65D 90/023; C12M 23/20; B65D 90/022; C12M 23/02

(57) ABSTRACT

A large container having a rigid wall constructed of plates and an inner, fluid-impermeable film that covers the inner surface of the plates, so as to provide a sealed interior space of the large container. Multiple sections of film are used to cover the plates, either as a piece of film that is provided on each individual plate or as a larger sheet that extends over a plurality of plates. The pieces of film are dimensioned such that adjacent sections of film have an overlap section, so that overlapping sections can be affixed to each other in a manner that forms a fluid-impermeable sealed seam.

### BACKGROUND INFORMATION

### Field of the Invention

The invention relates to a so-called “large container” that is used in various fields of endeavor. More particularly, the invention relates to a large container that is used in agriculture and in biogas production.

### Discussion of the Prior Art

German document DE 86 33 074 U1 discloses a large container that is conventionally used in biogas production. In practice, such large containers are constructed with a plurality of enameled steel plates. The enamel is intended to protect the steel material against chemical attack by the material that is to be stored in the container. Joints between abutting plates have to be sealed and for this reason assembling the large container is a comparatively costly process. Also, depending on the type of seal that is used, assembly may be dependent on weather conditions. For example, it is often the case that sealing can be done only under certain external weather conditions.

Another disadvantage of the conventional large container is that the protective layer on the steel plates, i.e., the enamel coating, is limited in the amount of mechanical loading it can withstand, and this has to be taken into consideration during assembly and also later during operation.

It is known in the industry to use large containers that do not have enameled steel plates, but instead, have stainless steel plates. This provides the desired the chemical resistance, without using the mechanically sensitive enamel coating. The material cost for these stainless steel plates is relatively high. Also, the joints between the plates still have to be sealed with these stainless steel large containers, so the costs of the containers remains high despite the fact that the plates do not have to be coated.

It is also known in the industry to use large containers that have walls made of concrete. Consistent quality is ensured by prefabricating the concrete plates for the walls in a factory setting. This reduces or eliminates the difficulties that can be caused by weather conditions that are adverse to fabricating and assembling the conventional large container directly at the installation site. Nevertheless, providing a seal between the individual prefabricated plates and the relatively high transport costs have a negative impact on the production costs of such concrete large containers. This is particularly true, if the containers from a European manufacturer are to be installed on other continents, such as, for example, Australia or North America.

Unconventional large containers are known in the industry, in which at least the wall or possibly the wall and floor are manufactured monolithically, using in-situ concrete. The availability of such large containers depends directly from the availability of the necessary forms for poured concrete. In this case, too, the transport costs for the forms have a significant impact on the manufacturing cost for these types of large containers. The quality of the finished in-situ concrete container may also be subject to fluctuations, depending on the care taken by the assembly personnel, the availability and/or the quality of the aggregate that is used in each case, and the volumetric composition of the concrete.

What is needed, therefore, is a large container that can be assembled quickly, in an uncomplicated manner, and independently of external weather conditions. What is further needed is such a container that can be manufactured and assembled as economically as possible, and that facilitates trouble-free operation of the large container, particularly in the field of biogas production.

### BRIEF SUMMARY OF THE INVENTION

It is an object of the invention to provide a large container that is constructed with a plurality of wall plates of sufficient strength and rigidity to provide the necessary static framework for use in biogas production. It is a further object to provide such a large container that has an effective seal between the interior storage space and the wall plates, whereby achieving the seal is not dependent on favorable weather conditions at the time of installation.

The large container according to the invention is constructed of a plurality of wall plates and with a fluid-impermeable film liner that is applied to the inside surface of the plates and that provides a seal between the interior storage space of the container and the wall plates. In other words, the inside of the wall of the large container is covered with a film that is resistant to the chemical properties of the material to be stored in the container. The complete film liner is constructed from a plurality of film pieces or sections that are adjoined to each other in such a way as to create the fluid-impermeable film liner.

The use of this film eliminates the need to use wall plates that are made of a material that has the necessary chemical resistance. The wall plates, then, have to provide the necessary mechanical stability for the container and also provide protection against the elements of the weather, but, because the plates themselves do not have to provide chemical resistance against the stored material, they do not have to have an enamel coating, which is brittle, or be made of costly stainless steel.

A further advantage of the large container according to the invention is that joints between the abutting plates do not have to be sealed. The film that is used to line the large container is, for example, a synthetic film that is impermeable to fluids, i.e., liquids and gases. With this film liner, liquids may be stored securely in the large container without chemically attacking the material of the plates. Because the film is also gas-impermeable, gases escaping the liquid are also not able to permeate the film and damage the plates.

According to the invention, the film is fixed in placed inside the container. Thus, the lining is not a freely movable “film sack” that is suspended loosely inside the wall of the container, but rather, constructed of a plurality of film sections that are sealingly affixed to each other and also fixed in place inside the container. This is important to reduce the possibility that flow movement of the material stored within the large container can cause the film to shift or deform. Fixing the film in place reduces the chances of local overloading on the film, which could result in tears in the film or a shifting of the film. The film is thus effectively prevented from sliding into a discharge opening and possibly plugging it, or coming into contact with agitation equipment or other equipment that is installed within the large container.

In a first embodiment of the large container according to the invention, the individual wall sections or plates are prefabricated at the factory as composite elements. The construction of the plates allows for a high degree of prefabrication of the large container, independent of external weather conditions. The side of the plate that, when assembled to the large container, will subsequently be facing the interior of the container is referred to as the interior face of the plate. In this embodiment, the film is applied to the interior face at the factory with a film. In this first embodiment, the individual film sections are part of a multi-layer construction of the wall plate and the film section is fixed in place on the plate.

Only the individual film sections of the individual plates have to be sealingly bonded with each other, in order to create the overall film. One of the issues with using a putty-like sealing compound that is typically used to seal the joints between the individual steel plates is that the compound has to be applied under certain ambient conditions, such as temperature and dryness. Creating a seal between film sections by welding or fusing is a process that is much less sensitive to various weather conditions.

In a second embodiment, the container wall is assembled on site, without requiring the use of prefabricated, multi-layer wall plates. Instead, an outer layer of the wall is erected, which defines the statics of the wall, and the film sections are then installed inside this container wall and sealingly bonded with each other, for example, welded or fused. Fixing these film sections is done with the help of film strips that are fastened mechanically to the rigid outer plates of the wall, for example, with the help of screws and large-area washers, or even larger retaining plates. The mechanical fasteners securely fasten the film strips to the outer rigid plates of the wall construction.

The fasteners may also cover a relatively large area, so as to distribute loading and thereby avoid tearing of the film strips under load, and to reduce the relative movement between film strips and the fastening means that the fastening inherently exerts. It is necessary, though, that even when the mechanical fasteners cover a relatively large area on the film strips, they do not cover the entire surface area of the film strips. The mechanical fasteners may have cut-outs, for example, in the form of so-called windows or openings, through which contact with the particular film strip is possible. Ideally, the film strips have larger dimensions than those of the mentioned mechanical fastereners, so that the film strips extend out beyond the fasteners. An inner film is assembled after the film strips have been assembled. This inner film may be bonded with these film strips at places where the film strips are still accessible, for example, there, where the strips extend beyond the mechanical fasteners, so that even with this embodiment, the film is securely placed on the wall of the large container in a way that prevents the film from shifting.

Adjacent film strips may be fused with each other, for example, by applying heat or cold to achieve fused, sealed seams. Or they may be adhesively affixed to each other, or pressed tightly against each other by means of clamp strips, or fastened with other known suitable means. Just by way of example, the discussion below assumes a welded or fused seam, which has proven effective in practice, even with other applications of film.

Also the work of sealing may be done independently of building the container structure. In other words, the large container may first be erected and the sealing work, namely, welding the seams on the film sections, may be done later under suitable weather conditions. Placing the film on the protected inner side of the wall means that the film sections can be welded in dry conditions and possibly in a heated inner space of the large container. This is particularly an advantage when the installation of the large container is to be completed as soon as possible and for this reason the sealing work has to be done immediately, even under weather conditions that are not ideal.

The large container according to the invention is very suitable for use as a fermenter in biogas production, because of a construction that uses plates that provide the necessary mechanical stability and then a film that provides excellent protection of the plates against chemical attack from the material stored in the container. The large container is also suitable for storing acidic materials, for example, turnip mash, which has a pH value of approx. 2.5 and therefore creates such a high demand with regard to chemical stability that even plates made of a relatively expensive material, such as a V2A stainless steel, are not suitable for such use. In addition to its chemical resistance, the film also allows an optimal seal to be achieved quickly and reliably. Thus, the large container may be made impermeable to gases, which is necessary for a fermenter or for storing fermentation residue in the field of biogas production.

The individual sections of film that form the overall inner covering of the large container may be arranged such that they don't necessarily overlap each other. For example, a film piece of the above mentioned first embodiment may have the same dimensions as the particular plate to which the film piece is affixed, or the piece may even have slightly smaller dimensions than that of the respective plate. Film pieces directly adjacent to each other may then be welded to each other simply by placing a film strip over the joint between the two adjacent film pieces and welding the strip to both film pieces.

A first particularly advantageous aspect of the construction according to the invention lies in the fact that, in order to create the adhesive and/or sealing seam, overlapping film sections or film strips or film pieces have a sealing plane between them, that, on a circular container, runs parallel to the wall surface, that is, runs tangential. The inner pressure prevailing in the container, for example, the pressure resulting from the weight of the material stored in the container, presses the overlapping surfaces of two film sections or of a film strip and a film section against each other, and thereby improves the sealing effect of the sealing seam. By contrast, thick-walled plates, such as precast concrete elements, are assembled to abut each other, rather than overlap, and the prevailing pressure inside the container tends to press these plates apart, i.e., does not reinforce the seal between the plates, but rather, weakens it.

Due to the relatively thin wall thickness of the plates used in the large container according to the invention, the plates may be assembled in an overlapping arrangement, and then fastened to each other by means of mechanical fasteners. For example, the plates may be made of metal and be screwed to each other in the overlap area.

The overlap areas of film also provide a means of covering the screw heads that extend into the inner space of the container. Thus, even the fastener elements of the plates may be optimally protected against chemical attack by the liquid or gaseous material within the container and the mechanical stability of the large container be guaranteed over a long service life.

Instead of the above described indirect welding of film sections to each by use of a strip that is welded to two adjacent film sections, the film sections may overlap each other and be welded directly to each other. This overlapping construction of the film sections simplifies construction of the large container, because it eliminates the need to handle an additional film strip. Also, a single welded seam ensures a tight bond of two adjacent film sections, whereas the use of an additional film strip would require two such welded seams, namely, a seam with each of the two adjacent film sections.

In the overlapping construction of the first embodiment, which forsees the use of individual film sections on prefabricated, multi-layer wall plates, the film sections are dimensioned somewhat larger than the dimensions of respective plate, so that an overlap area of the film section extends beyond one or more edges of the plate sufficiently to overlap with the overlap area of an adjacent film section. Assuming a rectangular plate, the film may extend beyond all four edges of the plate, so that the layout of the plurality of plates that form the wall provides overall a roof-tile like overlapping construction of adjacent film pieces.

In the second embodiment in which the large container is constructed on site, rather than in a factory setting, the individual film sections may be constructed as long bands that extend over the entire height of the wall of the large container.

Basically, it is understood that additional material may be provided between the two overlapping film sections, for example, an intermediate strip made of a material different from that of the two film sections, whereby this intermediate strip is used to form a better weld with each of the two film sections than the two film sections with each other. Advantageously, however, the two film sections are welded directly to each other, because this single seam simplifies construction and also provides a reliable seal.

Advantageously, the wall plates of the wall for the large container may have dimensions that are greater than the dimensions that are customary in the industry, the customary dimension being maximum 3 m length and maximum 1.25 m wide, and typically 2 m×1.25 m. In particular, the plates may have dimensions of approx. 2 m×6 m. Use of these larger plates enables significantly faster assembly and also saves on material costs, because fewer wall plates are needed and, thus, the number of fastener elements, such as rivets or screws, needed to construct the wall is also reduced. Plates that are 2×6 m also fit without difficulty into conventional transportation means, such as trucks and containers.

In a first embodiment in which each plate is prefabricated at the factory with its own film section, the size of the plates has an effect on the length of the overall welded seams that have to be produced and that are needed to achieve the necessary seal, i.e., for bonding the individual adjacent film sections with each other. By contrast, in the second embodiment, the total length of the sealing seams to be produced may be less, because the individual film bands that are used extend across multiple plates.

There are several reasons for the limitation to the relatively small dimensions of the conventional plate, for example, 1.25 m×2 m: the enameled plates have to be protected, so that the enamel doesn't split off when the plates are handled. Also, the greater the dimensions of the enameled plates, the greater the danger that they will sag so much during transport and assembly, that cracks will occur in the relatively brittle enamel layer. Also, large enameled plates are so heavy that they can only be handled with the help of hoisting equipment. Hooks or similar hoisting equipment increase the risk of damaging the enamel coating.

The relatively smaller dimensions of the conventional enameled plates avoids cracking of the enamel coating, because the weight of the plate is reduced and, thus, the likelihood that the plate will sag. This lower weight also makes it possible to handle the plates manually, without the use of hoisting equipment and this gentler way of handling the plates reduces the risk of causing splits in the enamel.

The plates for the large container according to the invention enable the use of plates with larger dimensions and this has advantages that include faster assembly, reduced material costs, and a reduction in the total length of sealing seams, while at the same time eliminating the disadvantages of the conventional plate.

The wall plates for the large container according to the invention may be made of metal and, particularly, steel. Steel plates provide the advantage that the wall thickness may be relatively thin, compared to the thickness of a concrete wall plate, yet provide the desired mechanical strength and rigidity for the intended use. This reduced wall thickness means that the plates weigh less and the transport volume is less, and these are advantages that reduce transportation costs from a manufacturing site to the particular installation site.

Depending on the material used, it may be economically advantageous to use a metal which requires additional treatment in the form of corrosion protection, instead of using a higher-cost corrosion-resistant metal. Well known methods for protecting steel plates against weathering influences that are readily applicable and also economical may be used. One such method is to apply a coating. Virtually all color shades of the RAL color pallet are available, so that the outwardly visible coloring of the wall may be designed according to the desires of manufacturer or the operator of the large container. The coating may also be multi-layered, in order to achieve a particularly reliable weathering and/or corrosion protection and/or a highly versatile optical external design of the plates. An example of such a multi-layer coating may include a first inner layer that is a so-called cathodic dip coating and then a second outer layer that is a powder coating.

In one embodiment of the large container, the wall may be flanged onto a concrete floor plate that was poured in-situ and the joint between the film and/or the flange and the floor sealed by means of a sealing compound.

In another embodiment of the large container, the film may also cover the floor, for example, when it is desired that the floor have a particularly high chemical resistance against the material to be stored in the large container. In this case, the film lining of the large container may be configured in a sack-shaped manner by welding a corresponding bottom film to the wall film, thereby ensuring a sealing of the entire wall and floor area. This embodiment of the large container means that the floor does not have to have a particularly high chemical resistance, but rather that it only have the required load bearing capacity. For example, in a first embodiment, the wall may be placed as a multi-part steel ring on the ground. Typically, the ground is first graded to create a flat plane. The film is then introduced into this steel ring, so that the storage interior is sealed off against the floor and the wall.

In a variation of this in-situ concrete floor, the concrete floor itself may not have to be resistant to chemical attack, but rather, merely provide a sufficiently reliable anti-bite protection, in order to prevent damage to the film, for example, by rodents.

As mentioned above, the film layer on the large container according to the invention is fixed in place. In the first embodiment, each film section is fixed to the respective plate as an inner layer of a multi-layered sandwich-like structure and, in the second embodiment, the film is fixed to the plate by means of mechanical fasteners. In addition, it may also be advantageous to place the film over the surface of the floor and to fasten it at several points to the floor. A technique known in the field of roofing, for example, flat roof sealing that is used to fix roofing films in place, may be used for this purpose. Having a film fixed in place makes it possible to use an agitator in the interior of the large container. It also reduces the amount of flow energy that is transferred from, for example, flowing bulk material, to the film on the floor or the wall. Fixing the film in place ensures that the film cannot become detached from the wall or from the floor and, for example, slide into the mentioned agitator. Compared to simply suspending the film as a type of sack in the large container, fixing the film in place keeps the mechanical loading on the film as small as possible, and this reduces the risk of the film tearing or detaching from the wall or floor panels, and thus, reduces the risk of damaging the seal between the interior storage space and the container.

Depending on the intended use, a thermally insulating intermediate layer may be provided between the film and the wall plates and/or between the film and the floor. For example, when using the large container as a fermenter for biogas production, it may be advantageous to maintain a certain temperature level in the large container. The thermally insulating layer reduces heat loss, thereby increasing the efficiency of the fermenter. This intermediate layer may be preassembled at the factory, particularly when used with the first embodiment. For example, the intermediate layer may be affixed to the plates, so that the large container may still be assembled in an uncomplicated manner and quickly, even when a thermally insulating intermediate layer is used.

It may be advantageous to use two or more intermediate layers, instead of just a single intermediate layer. So, for instance, two thinner layers may be used, instead of a thicker single layer. For example, instead of using a thick textile, for example, fleece-like or other non-woven material, a harder, yet thinner, material may be used, such as, for example, expanded polystyrene (EPS) panels. If the EPS panels have a greater wall thickness, it cannot be ruled out that creases or kinks occur when the EPS panels are bent. If this should happen, full-surface contact between the insulating panel and the steel plate could be interrupted and this would create the risk of heat bridges or cold bridges, which impair the effectiveness of the desired thermal insulation. The outer plates, for example, steel plates, are curved according to the desired diameter of the large container. To provide insulating panels that are curved to conform to the radius of the wall plate would counter that risk, but would be economically disadvantageous. The use of two or more intermediate layers, which are relatively thin, allows the intermediate layers to be placed without creases or wrinkles against the outer steel plate, one intermediate layer over the other. This also means that the insulating intermediate layers may be flat sheets, thus improving the economic costs of the construction of the large container according to the invention.

Advantageously, the inner surface of the outer wall may be vented, in order to reduce corrosion problems on the inner surface of the wall plates and also to avoid entraining undesirable moisture into the thermally insulating layer. For this purpose, a so-called spacer sheet or band may be placed against the inside of the plates, to create a venting space. Materials are already known that are suitable to serve as the spacer, for example sheet-like materials, such as a fleece, a spacer fabric, i.e., nubbed film or the like. It is essential that the spacer create a convection space that allows ventilation on the inner side plates inside the container, so that condensation on this inner side can then easily flow downwards through the convection space or, on the other hand, if ventilation air is flowing through the convection space, can readily evaporate.

The use of a spacer sheet also makes it possible to construct the large container as a double-walled container, without the need to seal all of the joints between the outer plates. In this case, the spacer then has a layer that is impermeable to the fluid to be stored in the bulk container. The impermeability is provided in any case when a nubbed film is used as a spacer, because the entire nubbed film is made from a three-dimensionally shaped, fluid-impermeable material. The inner film of the large container, described above, forms the first impermeable container wall, and the layer of nubbed film forms a second impermeable layer. The nubbed film may also be impermeably bonded together, for example, adhesively bonded or welded, similarly to the method of sealing the film sections that form the inner film.

### BRIEF DESCRIPTION OF THE DRAWINGS

The present invention is described with reference to the accompanying drawings. In the drawings, like reference numbers indicate identical or functionally similar elements.

FIG. 1 shows a vertical, partially broken away section through the wall area of a first embodiment of a large container according to the invention.

FIG. 2 shows a vertical section through the wall area of a second embodiment, illustrating the use of an inner fluid-permeable layer against a thermally insulating intermediate layer and an outer fluid-impermeable layer between the outer surface of the insulating layer and the wall plate.

FIG. 3 illustrates a wall segment having a film layer with an overlap area for welding with an overlap area of an adjacent plate, an intermediate layer, a spacer layer, and a thin outer wall plate, and a U-shaped connector piece for connecting two outer wall plates.

FIG. 4 illustrates a plurality of wall segments connected to each other, with a film that is a single band that extends over the entire height of the wall segments.

FIG. 5 illustrates two wall segments, indicating a welded seam joining the film pieces of the two segments.

FIG. 6 is a side elevation view of two wall segments joined by a threaded fastener and showing the various layers that comprise the composite structure of the wall 3.

### DETAILED DESCRIPTION OF THE INVENTION

The present invention will now be described more fully in detail with reference to the accompanying drawings, in which the preferred embodiments of the invention are shown. This invention should not, however, be construed as limited to the embodiments set forth herein; rather, they are provided so that this disclosure will be complete and will fully convey the scope of the invention to those skilled in the art.

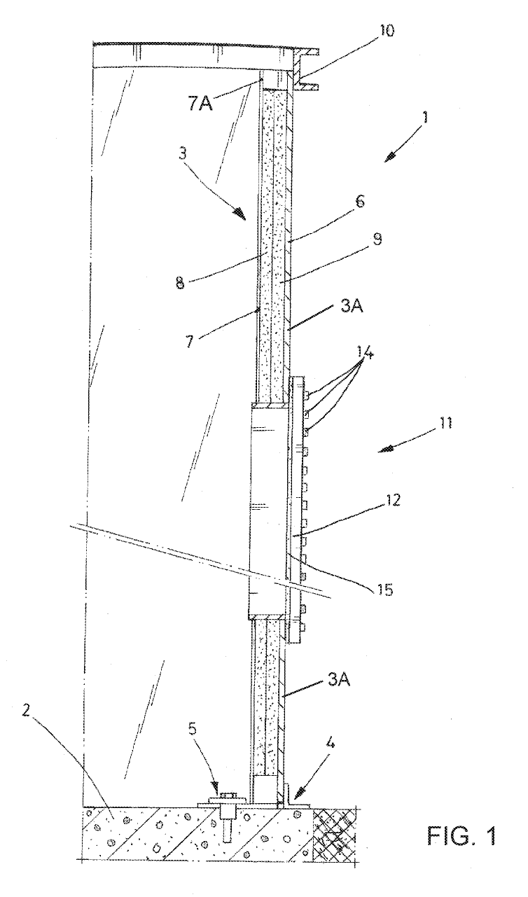

FIG. 1 is a cross-sectional view, showing a representative section of a large container 1 according to the invention. The large container 1 has a floor plate 2 made of in-situ concrete and a wall 3. A lower end of the wall 3 is flanged onto the floor plate 2. An L-shaped floor flange 4 secures the outer edge of the wall to the plate 2 and an inner flange 5 secures the inner edge of the wall 3 against the floor plate 2.

The wall 3 is constructed from a plurality of wall segments 3A. As used hereinafter, the term “wall segment 3A” refers to a representative single element of the wall, and the term “wall 3” refers to the overall wall that encompasses a plurality of wall segments 3A. Each wall segment 3A comprises a wall plate 6, which in this case is made of steel and is protected against weathering influences by means of a coating, for example, a powder coating. A film 7 is provided on what is the inner surface of the plate 6 after the plates are assembled to form the wall. The film 7 is made of a plastic film that is suitable to withstand any chemical attack that may be exerted by the material to be stored in the large container 1, and preferably, depending on the stored material, is a film that is highly resistant to acids.

As can be seen in FIG. 1, a first intermediate layer 8 and a second intermediate layer 9 are sandwiched between the film 7 and the wall plate 6 to complete the wall segment 3A. These intermediate layers 8 and 9 are thermally insulating layers. The film 7 is fixed in place on the first intermediate layer 8, and the second intermediate layer 9 is affixed directly to the plate 6, for example, adhesively. The first and second layers 8, 9 are also affixed to each other, for example, adhesively. These intermediate layers 8 and 9 are constructed of expanded polystyrene (EPS) and may be identical with regard to layer thickness and density, as well as to other material properties.

In this first embodiment, both the film 7 and the plate 6 extend upward beyond the intermediate layers 8 and 9 to create a wall segment 3A that may be readily joined with another wall segment 3A. A second wall plate 6 may be placed on the upper end of the wall plate 6 and the two plates 6 fastened by some mechanical means, such as a flange or bracket 10. Shown in the drawing is a U-shaped connecting flange 10 that extends along and is fixedly fastened to the lower plate 6, for example, welded or screwed. The connecting flange 10 extends upwards beyond the plate 6 so that it may be fastened in the same way to the lower edge of the next plate 6 that is placed on top of the one shown in the drawing.

A maintenance opening 11 may be provided on one or more the wall segments 3A, shown schematically in FIG. 1. This opening 11 is closed by means of a closing plate 12 that is fixedly, yet detachably fastened to the plate 6 by means of a plurality of threaded fasteners 14. A seal 15 is provided between the closing plate 12 and the plate 6, in order to ensure the impermeability of the maintenance opening 11. This seal 15 may be made of the same material as the film 7, but alternatively, may be formed by a putty-like sealing compound, which then has to be re-applied after the closing plate 12 has been removed and each time before the closing plate 12 is re-assembled on the wall segment 3A. Alternatively, the seal 15 may be constructed as a multi-use reusable seal 15, for example, in the form of a sponge rubber seal.

Sealing the maintenance opening 11 serves primarily to provide protection against the ingress of moisture from the outside, for example, due to precipitation or humid conditions. The film 7 in the inner chamber of the container 1 is completely sealed, so a pipe stub that extends from the maintenance opening 11 into the inner chamber may be provided, whereby a sealing ring made of an elastomeric material is easily provided on the stub on either side of the film 7. A preferred embodiment of the sealing arrangement is a press ring seal or annular space seal which may be compressed in the axial direction and which then expands radially to create a radial seal that presses sealingly against the pipe stub.

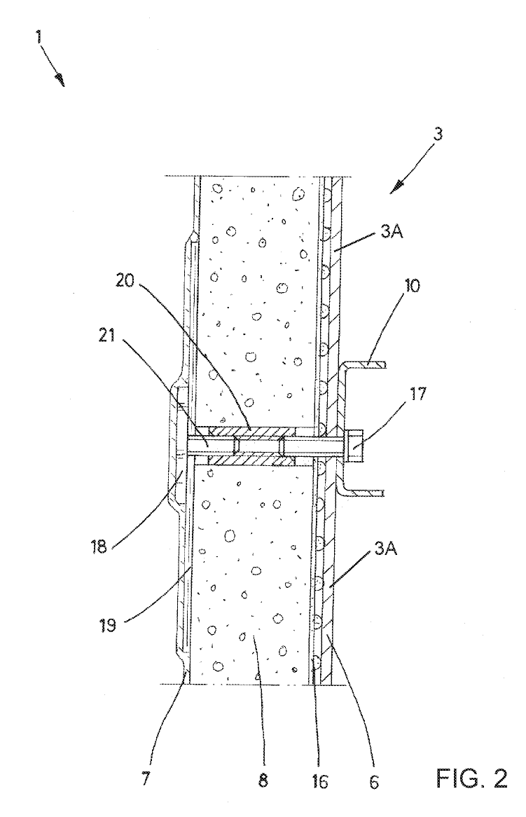

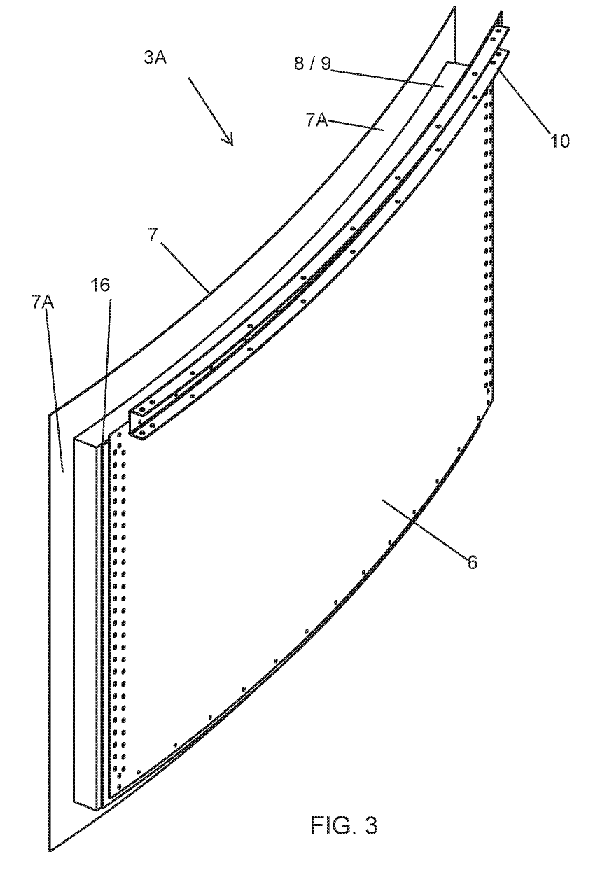

FIG. 2 illustrates two adjacent wall segments 3A of a second embodiment of the wall 3. In this embodiment, the intermediate layer 8 is provided as a thicker foamed panel, in contrast to the two thinner layers 8 and 9 shown in FIG. 1. In addition to the thermally insulating intermediate layer 8, however, a spacer sheet 16, is placed between the plate 6 and the intermediate layer. The spacer sheet 16 shown in this embodiment is a nubbed film 16 that has a planar base that lies up against the intermediate layer 8 and nubs that project away from the base and make contact with the plate 6. The nubs create spaces, i.e., convection spaces, that border the plate 6 and that aid in ventilation and thus also serve as a corrosion protection for the plates 6 of the wall 3, which are preferably made of metal.

Because of the large dimensions of the large container 1, a plurality of separate sections of spacer sheets 16 are used, due to the large size of the large container 1. These nubbed films or spacer sheets 16 may also be referred to as panels. These sheets 16 are welded together to form a layer that is impermeable to the fluid to be stored in the large container 1. The large container 1 according to FIG. 2 satisfies particularly stringent safety requirements because it is constructed as a double-walled container in that it has two separate sealing planes that are produced respectively by the sections of the film 7 that are welded together to form an impermeable layer and by the sections of the spacer sheets 16 that are also welded together to form an impermeable layer.

In the embodiment shown in FIG. 2, the film 7 is affixed to the wall 3 so that it cannot shift or slip in its position relative to the plates 6. Screws 17 extend in the region of the U-profiles 10 from the outside of the wall 3 to the inside of retainer plates 18. The retainer plates 18 not only hold the nubbed films 16 and the inner intermediate layer 8 in position on the plates 6, but also fix the position of film strips 19 that are arranged between the intermediate layer 8 and the film 7. The film strips 19 project beyond the retainer plates 18 and are welded to the film 7 in this projecting area, thereby fixing the film 7 more securely against the plates 6.

Threaded sleeves 20, each of which receives a screw 17, are affixed to the retainer plates 18. The threaded sleeves 20 compensate for variations in the length of the screw connection which arise due to the tolerances in the overall thickness of the wall 3 that result from the multi-layer construction. The threaded sleeves 20 are open at both axial ends and are screwed onto holding screws 21, which in turn are fastened to the retainer plates 18, for example, are screwed, adhesively affixed, or welded to a retaining plate 18.

The embodiment of FIG. 2 allows the wall 3 to be constructed directly at the installation site, without using pre-fabricated multi-layer wall sections. Rather, the plates 6 are first assembled to form the rigid outer wall. The spacer sheets 16, i.e., the nubbed films, are then welded together and the thermally insulating intermediate layers assembled from the inside of the structure formed by the outer wall. The composite structure of the wall 3 is then secured by assembling the retainer plates 18 and securing them in place with the screws 17. Subsequently, the individual sections of the film 7 are placed in the interior of the container by, for example, unrolling appropriately long sections or bands of the film 7 from the upper edge of the wall 3 downwards to the bottom of the wall 3. The sections of the film 7 are fixed by welding them to the areas of the film strips 19 which extend beyond the retainer plates 18 and also welding overlap areas of adjacent edges together.

FIGS. 3-7 illustrate details of the composite structure of the wall segment 3A and various ways of creating a sealed inner lining of the large container according to the invention. Each wall segment 3, when assembled, includes the wall plate 6, the spacer film 17, at least one intermediate layer 8, the liner film 7, and a fastening bracket 10.

FIG. 3 illustrates an embodiment of the wall segment 3A in which the film 7 is dimensioned to have an overlap area 7A that extends beyond the four sides of the intermediate layers 8 and/or 9 and wall plate 6. This overlap area 7A is provided so that the overlap areas 7A of two adjacent wall segments 3A overlap each other are then welded or fused together to form a sealed seam.

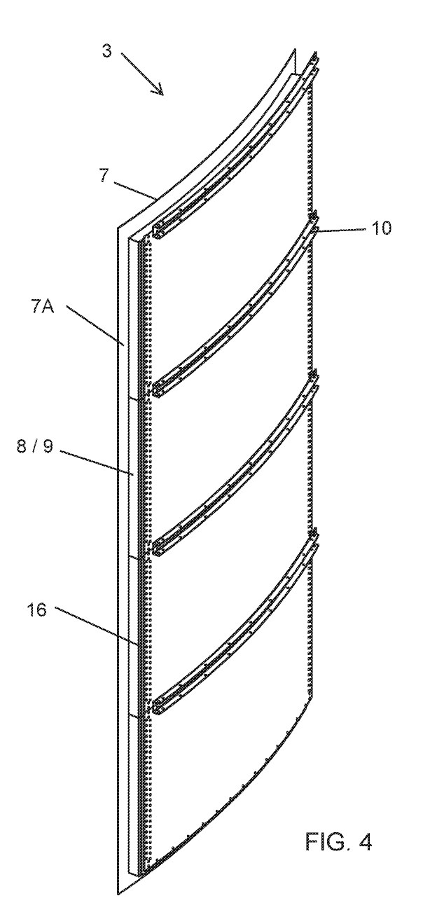

FIG. 4 illustrates several wall segments 3A assembled to form a vertical section of the wall 3. In this illustration, the film 7 is provided as a long band that extends over the entire height of the several wall segments 3A and has overlap areas 7A along its edges for creating welded seams with adjacent pieces of film.

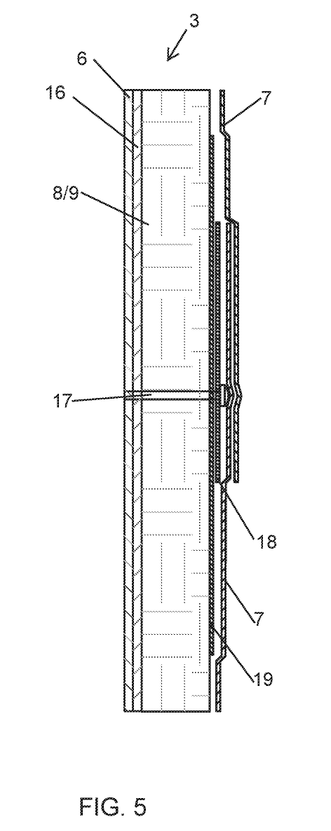

FIG. 5 is a side elevation view of a section of the wall 3, illustrating the double-wall construction of the large container. The wall 3 comprises overlapping films 7, the retainer plate 18, the film strip 19, the intermediate layer 18, the spacer film 16, and the outer wall plate 6. The film 7 and spacer film 16 each provide a sealed plane, thereby creating the fluid-impermeable double-wall construction.

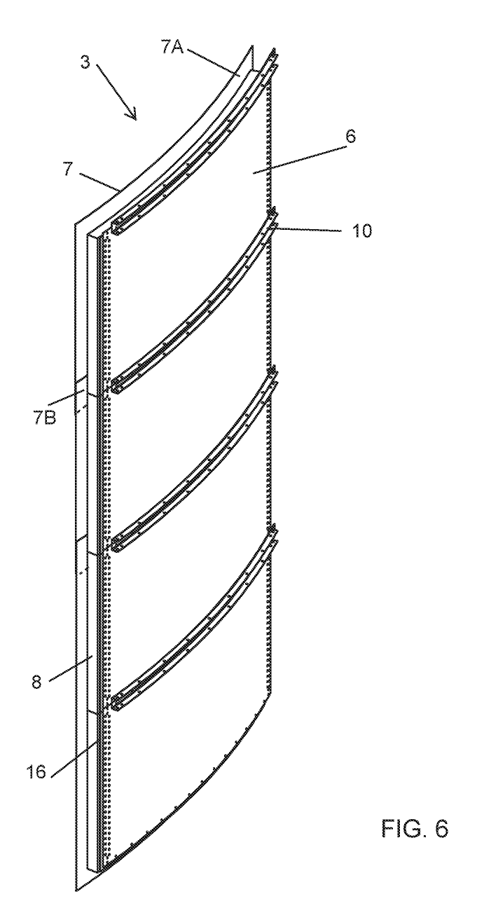

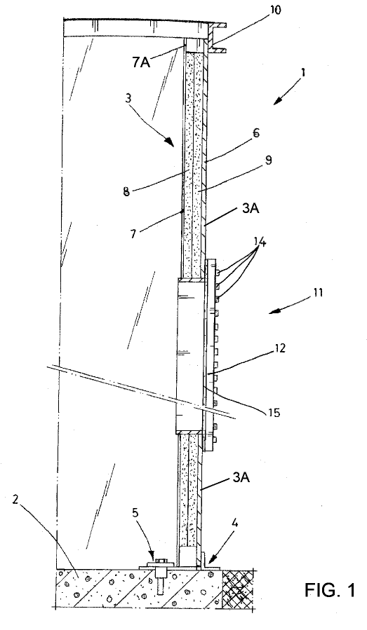

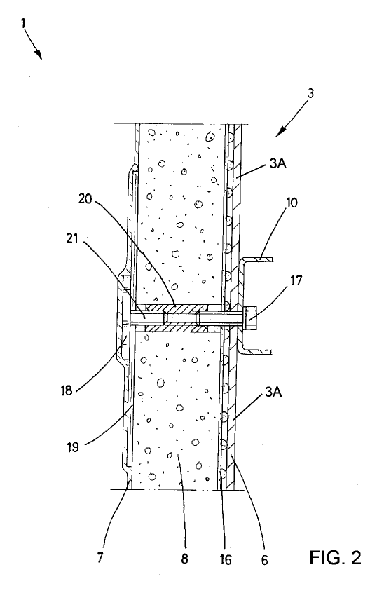

FIG. 6 is similar to the illustration of FIG. 4, but in this embodiment, the film 7 is shown constructed from two film pieces, each of which is dimensioned to correspond to a single wall segment and a film piece that is dimensioned to extend across a plurality of wall segments.

It is understood that the embodiments described herein are merely illustrative of the present invention. Variations in the construction of the large container may be contemplated by one skilled in the art without limiting the intended scope of the invention herein disclosed and as defined by the following claims.

What is claimed is:

1. A large container comprising:
a floor;
a wall assembled from a plurality of plates, the plurality of plates having an inner surface that faces an interior of the wall after it is assembled; and
a film that covers the inner surface of the plurality of plates, the film provided as a plurality of film sections;
wherein a sealing seam that is impermeable to fluid is formed between every two adjacent joints of the film sections to form a completely sealed film.

2. The large container of claim 1, wherein the plurality of film sections are provided as separate sections, one for each plate of the plurality of plates, the film sections being dimensioned larger than each respective plate; and
wherein the plate and film are provided as a prefabricated wall segment.

3. The large container of claim 1, wherein the film sections are dimensioned to extend over several plates of the plurality of plates.

4. The large container of claim 3, wherein the wall has an overall height and the film sections are dimensioned to extend over the overall height.

5. The container of claim 1, wherein adjacent film sections on the wall overlap each other with an area of overlap and are welded directly to each another in the area of overlap.

6. The large container of claim 1, wherein one or more of the plurality of plates have a height of greater than 3 m.

7. The large container of claim 6, wherein one or more of the plurality of plates have a height of greater than 6 m.

8. The large container of claim 1, wherein one or more of the plurality of plates have a width of more than 1.5 m.

9. The large container of claim 8, wherein one or more of the plurality of plates have a width of 2 m.

10. The large container of claim 1, wherein each plate of the plurality of plates is made of steel.

11. The large container of claim 1, wherein the each plate of the plurality of plates has a surface coating.

12. The large container of claim 1, wherein the floor is also lined with the film.

13. The large container of claim 12, wherein the film is affixed to the floor at a plurality of points on the floor.

14. The large container of claim 12, wherein a thermally insulating intermediate layer is provided between the film and the floor.

15. The large container of claim 1, wherein a thermally insulating intermediate layer is provided between the film and the plurality of plates.

16. The large container of claim 15, wherein the thermally insulating intermediate layer includes two intermediate layers.

17. The large container of claim 1, wherein a spacer sheet is provided between the plurality of plates and the film, thereby creating a ventilation space.

18. The large container of claim 17, wherein the spacer sheet has a fluid-impermeable layer, and wherein the fluid-impermeable layers of adjacent spacer sheets are connected to each other so as to provide a fluid-impermeable barrier against the fluid to be stored in the large container.
